(19) United States
(12) Patent Application Publication
(10) Pub. No.: US 2008/0213904 A1
(43) Pub. Date: Sep. 4, 2008

(54) Monitoring drug compliance, food-intake or toxin-intake using non-invasively-read labels

(76) Inventors: John W. Sliwa, Los Altos Hills, CA (US); Herbert L. Berman, Los Altos Hills, CA (US); Bryan T. Oronsky, Los Altos, CA (US); Carol A. Tosaya, Los Altos Hills, CA (US)

Correspondence Address: David W. Collins;Intellectual Property Law, Suite 100, 512 E. Whitehouse Canyon Road, Green Valley, AZ, 85614, US

(21) Appl. No.: 11/895,400

(22) Filed: Aug. 23, 2007

Related U.S. Application Data
(60) Provisional application No. 60/840,281, filed on Aug. 24, 2006.

(51) Int. Cl. G01N 33/00; G01N 37/00; G01N 21/64; F21K 2/00

(52) U.S. Cl. 436/56; 436/164; 436/172; 250/461.1

(57) ABSTRACT

A system is disclosed for monitoring a property of an ingested or in-taken drug, food, drink or toxic substance, non-invasively or minimally invasively, which can also identify the subject person being monitored, if desired. The system comprises: a means of labeling the substance with a labeling media to have a useful signature indicative of, or bearing a relation to the property; a means to allow the signature to be read non-invasively or minimally invasively; and a means to identify, in any manner, who is being monitored.

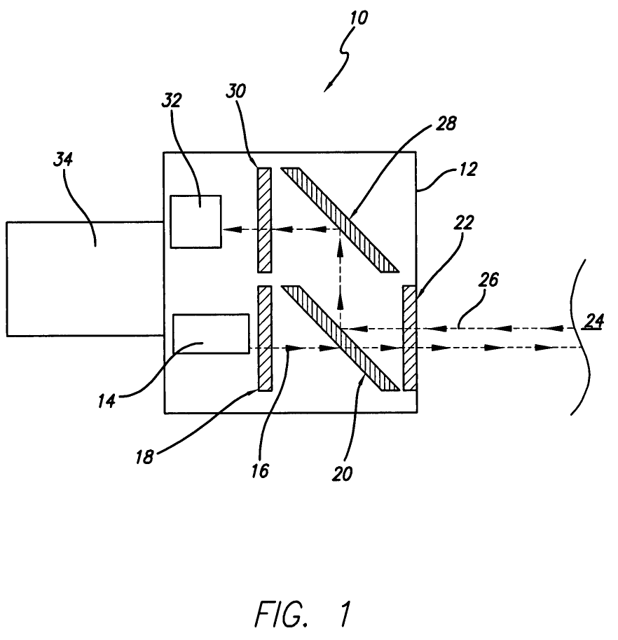

### CROSS-REFERENCE TO RELATED APPLICATION

The present application claims priority from provisional application Ser. No. 60/840,281, filed Aug. 24, 2006.

### BACKGROUND OF THE INVENTION

It would be desirable to be able to easily and non-invasively monitor what goes into our bodies-intentionally or unintentionally. A first example of this, which will be our primary teaching vehicle, will be that of drug-intake. Increasingly it is becoming important to be able to enforce drug-administration compliance and to be aware of compliance violations. A similar argument exists for performing improved monitoring of clinical trials. Similarly, readers will be aware that one could also use the invention to prevent incompatible medications from being simultaneously utilized.

A second general area of intake-monitoring is that of food and drink. At the present time there is no method or apparatus for an individual to detect or measure what he/she has ingested in terms of types, quantities or caloric-content of the infinite variety of foods and drink he or she consumes. At the same time obesity and obesity-driven diseases are reaching epidemic proportions. Food and drink might include banned substances and illegal drugs.

A third general area of intake-monitoring is that of undesired or harmful toxics-intake by way of accidental or criminal exposure. Such toxics could be anything from heavy metals, asbestos or PCBs. They might even be inhalation of industrial fumes or particulates.

A fourth area involves pulmonary and intranasal delivery of medications such as corticosteroids, bronchodilators and anticholinergics for conditions such as asthma and chronic obstructive pulmonary disease.

A fifth area of intake monitoring involves the detection of adulterated or fake drugs by labeling the real medicine in order to determine whether unauthorized substitution has occurred.

In all of these diverse applications, some voluntary and some enforced, we essentially pretag or prelabel the ingestible or absorbable/adsorbable drug, food or material which will eventually be intentionally or unintentionally ingested. Note that the inventive scope includes all intake routes into the body such as ingestion, eating, drinking, inhaling, entry into a natural orifice, injection, etc.

The technology taught herein is applicable to all three of these broad application areas. We will primarily use the prescribed-drug compliance example to describe our invention as it is clearly a large potential market.

Poor patient compliance with prescribed medications is a widespread problem which may limit the effectiveness of many pharmacologic therapies and increase the likelihood of medication switching, physician visits or hospital admissions, which in turn raises healthcare costs. Compliance with prescribed treatment is a crucially important factor in the management of many chronic conditions such as diabetes mellitus, hypertension, alcohol dependence, epilepsy, schizophrenia, bipolar disorder, HIV and TB and acute therapy situations such as antibiotic treatment of infections. In many of these conditions, the patients take multiple medications at different intervals which may lead to confusion or forgetfulness as to what medication was taken and when. Patients who either fail to take their medications or who consume too much because they are not aware of their last dose of drug are potentially at risk for occurrence of the disease, relapse or recurrence and toxic overdose, respectively. This also includes the routine or intermittent use of prophylactic medications such as oral contraceptives, aspirin, 5-alpha reductase inhibitors, anti-malarials, statins, and migraine therapies as well as vitamins and minerals.

The consequences of poor compliance can thus be serious not only for the patient's health in terms of treatment failure, drug resistance, and drug toxicity but also for the public health as a result of increased drug resistance in the community and prolonged communicability through failure to eliminate the disease. Non-compliance is also particularly detrimental to clinical trials by introducing a major risk of bias in the interpretation of the results. Thus, the implications of poor drug compliance are enormous, and preventing this major factor of therapeutic failure is a paramount challenge. Improved compliance can optimize therapeutic benefit and result in better patient outcomes, more accurate data from clinical trials and substantial cost reductions.

Many prior art methods applied to monitor drug compliance can be hampered by serious shortcomings. Thus, pill-counts are unreliable, drug monitoring of the pre-scribed agent itself is dependent on the availability of sensitive assays, and often requires repeated blood or urine sampling along with a trained technician to perform the analysis, and most electronic monitoring is expensive and non-specific in terms of determining what type or what concentration of medication has actually been ingested. In contrast to the shortcomings of these methods, the present invention attempts to fulfill the necessary requirements for analyzing compliance as it provides an optical or visual, relatively-inexpensive means for patients or third parties to determine and document certain parameters associated with medication, food and toxics intake.

An example of a prior art device, described below, involves a wristwatch-like device worn by the patient. The “watch”, is a bidirectional optical transdermal sensor that transdermally detects fluorophore labels placed in ingested matter such as medicinal pills or medicinal capsules. This prior art device directly contacts the patient's skin all day, and perhaps all night long. The device is capable of, for example, alerting a remote doctor of noncompliance and alerting the patient to that noncompliance.

A wristwatch is a small form-factor device. What this means is that the space available for providing the needed bidirectional sensor and its necessary power-source or power-supply is quite limited. This necessarily means that that the S/N or signal-to-noise ratio will be inferior to that of a device which can take up more space, have a larger sensor and have a more powerful and longer-lasting power source.

### BRIEF DESCRIPTION OF THE DRAWINGS

The sole FIGURE is a schematic diagram of apparatus employed in the practice of the invention.

### DETAILED DESCRIPTION

Our invention preferably, but not necessarily, uses a sensor device which falls into at least one of these forms:
a) It is part of or integrated with a biometric device.
b) It is a peripheral of on integrated part of a PDA, cellphone or other personal electronic device.
c) It is a contact-free device, not requiring skin contact.
d) It is a skin-contact device but the contact is momentary.
e) It is a worn skin-contact device (much like the cited prior art, for example).
f) It is a device that utilizes a transdermal optical window or port.

In addition to these inventive device forms, we also teach a labeling material system for use with numerous types of new and prior art optical transdermal sensors. This labeling system utilizes nanoparticles or microparticle technology to offer a wide array of uniquely identifiable optical signatures to uniquely mark different types of drugs, foods, toxins, etc.

We claim this nanoparticles or microparticle labeling material separately as well as with use of the above inventive device categories or forms. We also claim the device forms separately as well.

Let us first examine our inventive product forms:

### (A) Biometric Device-Based Sensor:

The point of this embodiment is that we wish to verify, know or learn the identity of the patient or subject in combination with administering or monitoring the drug. In this manner there can be no cheating by the patient and a legal record of drug intake (or non-intake noncompliance) is produced. Typical biometric devices of relevance would include, for example, fingerprint readers, finger vein-pattern readers, hand-scanners, facial recognition devices, iris pattern readers, voice recognition devices, retinal-vein readers, implanted-ID chip readers or a redundant combination of these. For each of these we can assure that our optical drug-detection sensing is done on a known patient or trackable subject. It will not be possible to fake a test by having a friend scan his retina or finger. By knowing the “identity” we broadly mean both knowing the individual specifically as by name or patient-specific data OR simply knowing that the individual is the same (or different) individual as at a previous monitoring event or initialization, as would be possible knowing their fingerprint but not their name for example.

### (B) PDA, Cellphone, Music/Videoplayer, Dongle or Other Personal Electronic Device-Based or Device-Connected/Networked Sensor:

The point here is that increasingly, virtually everyone wears, carries or has close-at-hand a cellphone, PDA or similar personal electronic device. These devices, increasingly, have considerable computation ability, information storage ability, networking and data communication ability and location (GPS) monitoring ability. Further, they also increasingly have slots into which one can place or plug peripheral devices (like WiFi Modems), connectors into which one can plug apparatus such as earphones and microdisplays, and downloadable software and alerts. Our point here is to utilize as much of this capability as possible and not duplicate as in the prior art sensor. By this we mean, for example, the cellphone could use its battery to power the sensor, could use its processor to do any necessary sensing computations, use its communication abilities (1 or 2 way) to alert doctors or patients as necessary, or use its peripheral slot to house an adapter which is Bluetooth or cable connected to our sensor. Thus, in summary, at least a portion of the sensing process and/or its reporting is done by the cellphone or other personal electronic device. Obviously, the cellphone or PDA might also download drug-dosage, drug-interaction data or clinical-feedback from the monitoring entity. The cellphone or PDA may even contain software which acts as its own monitoring entity thereby eliminating or minimizing external communication needs.

### (C) Contact Free Sensor Devices:

By contact-free we mean that the patient or test-subject being monitored does not have to physically touch the sensor in order to have it perform its sensing function. To be explicit, by “touch the sensor” we mean the monitored subject bringing a bare skin or body surface to the sensor to take a skin-contact reading. Thus this configuration will always involve sensing light or optical, energy passing across a gap or space between the subject and at least some necessary sensor portion.

A preferred variation of this is that wherein optical energy passes from the skin (or eye etc) to the sensor (and/or vice-versa) across a gap, space or distance. This essentially is remote sensing of the test subject wherein the subject wears or has no sensor portion installed upon his/her body—not even temporarily. An example of this is that wherein a wall mounted sensor which emits an excitation laser beam or illumination (pulsed or CW) upon the patient and also has a photonic detector to receive responsive fluorescence indicative of drug concentration. All with no physical contact to the subject and perhaps working at distances of inches, feed, yards or even miles.

A second preferred variation of this is like the above using remote sensing—however the test subject or patient also, at least temporarily, wears or has an essential sensor component mounted on, against, in or in juxtaposition to his skin or body. So, for example, referring to the above example we could place an optical reference plate on the patient's skin (worn temporarily or worn long-term) which provides a fluorescence reference signal in order to do absolute measurements in the known manner. Note here that we now have an essential sensor portion (a signal reference) mounted or placed on the body but we still operate remotely or in a noncontact mode because the rest of the sensor is remote. The body worn or mounted sensor portion could be, for example, the said reference plate, an excitation illuminator or a photonic detector. In any event, needed sensor portions are resident both on the remote apparatus as well as on or juxtaposed to the patient. The reference article would likely be tamper-proof and would give an error message if tampered with. It might even be integrated into an item of jewelry, clothing or a temporary identification bracelet.

### (D) Momentary Skin-Contact Devices:

These are quite simple. Unlike the prior art worn watch these require only momentary contact-say from a fraction of a second to seconds. A sensor which most likely doubles as a fingerprint reader would accomplish identification and momentary-contact sensing. The device sensor may be mounted or carried anywhere-what is important is that contact is momentary and not uninterrupted for long periods. By fingerprinter reader we include all manner of such readers including those based on 1-D sensor arrays and 2-D sensor arrays as well as those already integrated in PCs, security systems, PDAs or other personal electronic devices.

### (E) Skin-Contact Devices:

What these have in common with the prior art is prolonged contact. We do suggest a few new forms including a shoe-mounted system, an ankle mounted system and an earlobe mounted system. However even with use of the prior art watch format our invention is novel when using our novel labeling means described below.

### (F) Transdermal Window Devices:

These sensors utilize an optical window placed-in or formed-in the skin. Such a window or port may be quite small and may be inserted by the patient himself—for example every few days or as-necessary. Typically, several sensed readings will be done for a given window mounted at a particular site. Such windows are known from the prior art and are typically infrared windows for sensing diabetic-related analytes being delivered by a pump or patch.

### Inventive Labeling Media and Methods:

(A) To begin with, we will call prior art excitation/emission systems as “additive” systems, as they create new spectral excitation peaks where there were none previously by introducing labeling media. We add a few new types of additive excitations over the prior art.
(B) Given that, we will also teach “subtractive” systems wherein the label media causes a removal of a previously existing peak or background emission, as by spectral subtraction or optical absorbtion.
(C) We also teach systems that utilize two energy forms for readout of the label. As an example the label may require optical excitation as well as excitation by an electrical, acoustic or magnetic field.
   Additive labels applicable to the invention:
1) Any known fluorophore or chromophore that is delivered into the body, regardless of whether it is simply mixed into a medication or is chemically bound to medication molecules. These include materials such as Rhodamine, chlorophyll from plant sources such as spinach, Indocyanine Green (IcG), Fluoroscein, and quinine. Other materials, and their excitation and emission wavelengths, are listed below in Table 1.
2) Any nanoparticles, typically ranging in size from a nanometer to hundreds of nanometers. These may be observed or detected in any manner including by any of: quantum dot effects, reflection, absorbtion, attenuation, refraction, polarization, electrical or zeta-charging, orientation, fluorescence, phosphorescence, any form of electronic excitation as drive by a driving electric, magnetic or electromagnetic field, magnetically, and/or capacitively.
3) Any microparticle, typically ranging in size from a fraction of a micron to several microns and having one or more of the detection means listed in (2) above.
4) Shaped or sized nanoparticles or microparticles whose size, shape or coating thickness(es) causes a specific behavior listed in (2), particularly at a specific frequency or frequencies.

The following Table I lists food-relevant fluorophors and their fluorescent properties:

TABLE I

| | Excitation | Emission |
|---|---|---|
| Fluorophor | Wavelength (nm) | Wavelength (nm) |
| Phenyl Alanine | 258 | 284 |
| Tyrosine | 276 | 302 |
| Tryptophan | 280 | 357 |
| Retinol | 346 | 480 |
| Riboflavin | 270 | 518 |
| Pryidioxin | 328 | 393 |
| A-tocopherol | 298 | 326 |
| NADH | 344 | 465 |
| ATP | 292 | 388 |
| Chlorophyll-a | 428 | 663 |
| Hematoporphyrin | 396 | 614 |

### Subtractive Labels:

These are new to the invention. Essentially, we create a background signal that is “subtracted from” by labeled media placed in the body. Think of it like seeing someone in a lit room through a drawn window shade. The bright window shade is our prearranged background and the person is the subtractive label that darkens (subtracts) from the bright shade.

This scheme is interesting because it can work in several different or simultaneous manners. As an example, one applies at least a first background media (the background signal) and then a label (in a drug for example) later inserted into the body chemically binds to or simply physically attenuates the background media emission, causing an optical spectrum change in the background material signature, such as by a local absorption due to the chemical bonding changes or attenuative blocking or scattering. The subtractive approach also offers more ways to deal with any food-based signals as the label and the background entities can be co-engineered.

A second version of this approach has a background material providing a background signal or spectrum and a label, by attenuation or fluorescent excitation, pulls energy out of the background spectrum which is detectable.

An advantage of subtractive techniques is that one can most easily arrange for the background and the label signals to be in spectral regions not blocked or interfered with by analytes not of interest.

Note that the background signal or spectrum is artificially created and not something that naturally preexists in the body, at least not with the desired controlled intensity and spectral distribution.

Say, for example, we want to uniquely label 12 drugs and monitor their concentrations. Using, for example, the nanoparticles or quantum dot approach, we can utilize 12 different sized label particles or 12 differently shaped label particles, one used to label each drug type. Each such particle type has a different optical excitation and/or emission signature as is known in the quantum-dot and nanoparticle fields. As is known, such particulates may be coated or uncoated and may or may not include binding receptors or ligands that cause the label to also have a biological or microbiological behavior.

Another scheme unique to the invention works as follows. A drug depot is introduced into the body. This might be one large implanted depot or may comprise slowly-dissolving particles that are stationary or move about in the circulatory system as they dissolve. One arranges for the undissolved depot drug or depot media to have a signature that is related to the amount of it still in the depot phase, for example the crystalline phase. Just as an example, one will get an X-ray diffraction signature from undissolved crystalline material but not from its dissolved atomic or molecular counterpart. X-rays can work through the skin and can be pulsed for minimal exposure. Likewise an X-Ray could cause a detectable fluorescence signal from the crystalline material in the depot.

The use of magnetic labels is particularly interesting since an external device can cause them to become oriented, if not locally collected. Oriented magnetic particles can be externally detected, as they act as an electrical component having a polarization, a polarization relaxation, an electromagnetic capacitive effect, etc.

We include in the scope of our invention the use of ultrasound to probe labels for a signature. As an example, label particles could have unique resonant frequencies (or antiresonant frequencies) that show up in a spectral scan as peaks or valleys. Micromachined labels, such as of silicon, glass or ceramic may be prepared. These may act as labels as by their physical resonances, by coating them with optically-distinguishable thin-film sandwiches, or patterning them with electromagnetically interactive microantennas.

In all of these schemes, we include the inclusion of reference labels as part of the labeling scheme. These are media that act to normalize or provide a quantitative reference for the signal so absolute concentration work can be done in a confident manner. However, unlike the prior art, which has reference materials on the external skin surface, we include having the reference labels being delivered into the body.

Included in the scope of our invention is the use of cavitating ultrasound delivered transdermally, for example. The idea here is that a cavitation event involved the generation of a momentary plasma microcavity that can be analyzed spectroscopically for optical signatures of the drugs (or drug fragments) we are trying to measure. Only one or a few such microcavitation events would be needed so that there is little or no net damage to tissue. The event may be a single-collapse event or a resonating bubble which does not completely collapse.

As mentioned above, also included in the scope of our invention is the use of labels that are micromachined or nanoengineered. By “micromachined” we mean that a lithography mask, mandrel or beam-writing scheme transfers a desired pattern from a mask, mold or mandrel or from electronic memory into the shape or pattern of the particle or into a label particle that has features on it or in it that give it a unique label signature. An example of this would be a microparticle having a pattern imprinted upon or in it that causes a predictable optical diffraction, polarization or interference effect. By “nanoengineering” we mean the design of custom molecules or molecular assemblages including molecular rods, tubes and sheets.

The present invention pertains to the monitoring of drug therapy and food intake based on optical detection of non-toxic near-infrared fluorophores coupled to ingested medicines or food. In contrast to prior art U.S. Pat. No. 6,663,846, entitled “Devices and methods for monitoring drug therapy compliance”, which employs transdermal devices to measure the emission from drug coupled fluorophores, the present invention relies on optical (preferably IR) imaging of the vasculature to optically detect the presence of the fluorophore in the blood. By “optical” we mean using photons and being human-invisible and/or human-nonvisible.

Pharmaceutical agents useful in the present invention include virtually any therapeutic substance swallowed, inhaled, injected, taken rectally or sublingually, transdermally absorbed including but not limited to: thrombin inhibitors, antithrombogenic agents, thrombolytic agents, fibrinolytic agents, vasospasm inhibitors, vasodilators, protease inhibitors, anti-tuberculosis agents, antihypertensive agents, antimicrobial agents, antibiotics, antifungals, antihelminths, anti-histamines, leukotriene inhibitors, inhibitors of surface glycoprotein receptors, antiplatelet agents, angiogenesis inhibitors, antimitotics, microtubule inhibitors, anti-secretory agents, actin inhibitors, remodeling inhibitors, antisense nucleotides, anti metabolites, antiproliferatives, antipsychotics, antidepressants, mood stabilizers, anti-epileptics, anticancer chemotherapeutic agents, anti-inflammatory steroid or non-steroidal anti-inflammatory agents, immunosuppressive agents, growth hormone antagonists, growth factors, dopamine agonists, hormones, anti-hormones, contraceptive agents, radiotherapeutic agents, peptides, proteins, enzymes, lipids, vaccines, drugs of abuse, extracellular matrix components, anti-ulcer, anti-migraine, analgesics, anesthetics, gases, anti-asthmatic, anti-malarial, anti-obesity, stimulant medications, free radical scavengers, chelators, antioxidants, anti polymerases, antiviral agents, photodynamic therapy agents, vitamins, minerals, herbs, and gene therapy agents.

One of the particularly useful properties of using near-IR (NIR) light is that oxygenated hemoglobin and deoxygenated hemoglobin both absorb in this region. As a result, when infrared light irradiates the backside of a region of the body such as the finger, a camera or detector with IR sensitivity placed on the opposite side is able to capture the image of the vasculature as shadows. If an IR fluorophore that is injected, inserted, coupled or coated onto a drug tablet or capsule is taken into the body, then the visual observation of a color or intensity change in the image of the vasculature captured by the camera or detector will provide visual confirmation to the observer that the drug or food has been ingested. Such a visual confirmation would particularly be useful to patients who have difficulty remembering whether a medication has been taken, often because the task is performed mechanically and does not register in their awareness.

Many patients also take a large number of medications, often prescribed for different intervals, and can easily become confused regarding whether any particular medication has been taken. As a result, an embodiment of the present invention relates to the creation of drug-specific signatures wherein particular drugs would be associated with specific colors, intensities, combination of colors or other suitable spectral features so that the observer would be able to determine which drug or drugs was taken; this is in contrast to the device described in U.S. Pat. No. 6,663,846, supra, which does not provide a mechanism for distinguishing between different drugs. These drug-specific signatures can be, for example, generated through the unique combination of different fluorophores and concentrations of fluorophores coupled or coated onto particular drugs. The observer would have access to a master template or chart, wherein particular drugs would correspond to specific colors, intensities, color combinations or spectral features. Alternatively, a reader incorporated into the device may be used, resulting in greater precision than human or visual interpretation of different colors, color intensities or spectral features.

In another embodiment of the present invention, the intensity of the signal generated may utilized to assess concentration of the fluorophore in the blood so that under or over dosage of the medication may be detected. This may include referencing it to a background marker or label. In another embodiment, drugs may be labeled for patient specificity. For example, aged couples who may have difficulty reading the labels on the pharmaceutical bottles may end up inadvertently taking each other's pills and exposing themselves to unintended and even dangerous drug side-effects. Patients may be supplied with droppers containing differently colored IR fluorophores to “label” their pills, in this way differentiating them from the pills of another person such as their spouse. In yet another embodiment, particular colors or intensities can serve as a distinctive warning of a strong or toxic medication.

Another embodiment of the invention involves the relatively inexpensive and straightforward way of detecting fluorescence at least in a relative manner through visible optical imaging, which in its simplest form uses neither ionizing radiation nor expensive sensory equipment to detect fluorescence. However, because of the limited penetration ability of visible light through the body, we envision testing such an optical device over preferential sites which are easily accessible such as the retina or sclera or where one might expect less scattering due to skin thinness such as the wrist, the eyelids, the digits, the nails, the penis and the scrotum. The skin or other preferential site is illuminated via an excitation filter with visible light of a specific wavelength (or wavelengths) which is absorbed by the fluorophore(s), causing them to emit longer wavelengths of light (of a different color than the absorbed light). The emitted fluorescence can be seen by the naked eye through the use of an emission filter(s) or detected by a sensory array such as a camera or photodetector with an emission filter. Since normal skin exhibits autofluorescence, it will be necessary to cancel out this background steady-state fluorescent signal. This could be accomplished, for example, with photographic overlay or even imaging analysis software if area-wise imaging is performed. By these means, an initial control image taken of the subject's skin could be subtracted from the image acquired after presumed ingestion of the medication coated with fluorophore to obtain an image that shows a fluorescence above baseline, indicating that the drug has been taken. The device might be handheld or “hands-free” consisting, for example, of glasses with a light source and filters to be worn either by the patient or examiner or of a row of lights with a filter that can be strapped over a certain area of the subject's skin such as the wrist.

An optical inventive device may consist in its most basic form of an NIR illuminator that irradiates a body part such as a finger, a detector in the form of a CCD or CMOS camera that captures the image and digital or analog screen for viewing. The device may also contain a reader to interpret the type of signal received and timer unit that is continuously running and internally maintains the current time. Again, one might utilize nonimaging photodetector technology as well. Optionally, the timer may be provided with a memory to store the viewing times for later retrieval. Auditory, visual or vibratory feedback can be provided to indicate not only that a fluorophore signal has been detected but also the type of signal detected corresponding to a drug-specific parameter or signature. Auditory feedback such as a tone can be provided by a miniature speaker. Visual feedback can be provided in the form of an image, a readout or a set of lights.

Imaging is attractive particularly if identity is to also be determined. However, imaging is not required for use of the invention and identification of the patient using the inventive means is also not required by the invention, and photodetector technology can also serve the measurement purpose. At any rate, the patient would probably be manually identified by a practitioner, nurse or by the user himself/herself as by keyboard input, ID Card or Dongle swiping or presentation or by use of a password unique to the patient.

An apparatus that is useful in the practice of the invention is shown in the sole FIGURE. The apparatus 10 includes a housing 12, within which is a collimated light source 14, which emits in the UV to blue region. Emitted light 16 passes through an excitation filter 18. An exemplary filter 18 is centered on 417 nm, with a bandwidth of 60 nm, such as a SEMROCK #FF01-417/60-25.

The light 16 then passes through a dichroic mirror 20, which passes short wavelengths (e.g., UV-blue) and reflects longer fluorescent wavelengths). The light is directed onto a fluorescent material 24, which fluoresces in the presence of the UV-blue light 18, returning a longer wavelength radiation 26, typically in the red regime.

The longer wavelength light 26 is reflected off the dichroic mirror 20 to a first surface mirror 28, through an emission filter 30, and onto an optical detector 32. An exemplary emission filter 30 is centered on 697 nm, with a bandwidth of 75 nm, such as a SEMROCK #FF01-697/75-25. The optical detector 32 is sensitive to the longer wavelengths, such as red.

A conventional power supply 32 provides appropriate power for the collimated light source 14 and the optical detector 32.

Also included in our inventive scope for use as labels is the use of atoms that have more than one unique nucleus (isotopes), such as isotopes of the same material having different numbers of neutrons. These are uniquely identifiable. One could even have a particular unique label be an atomic (or ionic) material that comprises a set ratio of two or more of its different isotopic forms. Given the use of isotopes and isotope-ratioed labels, there is a readily long list of possible stable labels available. These isotopes may, in some cases, be chemically combined into compounds and molecules and still detected as different isotopes. We include in the scope of our invention the use of slightly radioactive labels which have emission or decay signatures that can be detected-particularly those which have negligible natural background concentrations in the human body.

We also anticipate the use of the invention to monitor the action of environmental pollutants, toxics, and particulates on living entities such as plants, animals, and humans. Such harmful species might be industrially produced, naturally produced or criminally produced. Unlike present-day simple color-tracers or radioactive tracers, our media offer more unique identification possibilities and our devices are far more convenient to use and procure than expensive laboratory instrumentation used for that purpose today. Along these lines, the inventive device may have a “lab-on-a-chip” component which miniaturizes such present day instruments such as FTIR spectroscopes, for example.

What is claimed is:

1. A system for monitoring a property of an ingested or intaken drug, food, drink or toxic substance, non-invasively or minimally invasively, which can also identify the subject person being monitored, if desired, comprising:
a means of labeling the substance with a labeling media to have a useful signature indicative of, or bearing a relation to the property;
a means to allow the signature to be read non-invasively or minimally invasively; and
a means to identify, in any manner, who is being monitored.

2. The system of claim 1 wherein the identification means is biometric in nature.

3. The system of claim 1 wherein the identification means includes at least one of: a) a retinal scanner, b) a hand scanner, c) a facial-recognition system, d) a speech or voice-recognition system, e) any type of finger or hand scanner, f) a fingerprint scanner, g) a password, h) a memory dongle, i) an ID card, j) any type of person-unique optical signature, k) a vein pattern, l) any eye related pattern, or m) any type of implanted or body-attached ID chip, transmitter or transceiver whether removable by the wearer or not.

4. The system of claim 1 wherein the labeling media is or contains any one or more of: a) a nanoparticle, b) a quantum dot, c) a microparticle, d) an isotope, e) a fluorophore or chromophore, f) a micromachined or lithographically derived particle or member, g) any particle with a known size or shape, h) any particle with a known size or shape distribution, i) any particle with a known coating system, j) any particle with a functional microbiological coating, overlayer or outer surface, k) any particle having an optically interactive or unique thin-film or pattern, or l) any particle having an electromagnetically interactive unique signature, including a patterned microantenna.

5. The system of claim 1 being contained in, for use with, or being an interfacing or dependant peripheral of a PDA, cellphone, music/video player, dongle, ID card or other personal or personalized electronic product.

6. The system of claim 1 wherein the system allows for contact-free, spaced or distant monitoring of said substance.

7. The system of claim 1 wherein the system allows for momentary contact monitoring of said substance.

8. The system of claim 1 wherein the system allows for skin or mucous-membrane contact monitoring of said substance.

9. The system of claim 1 wherein the system allows for monitoring of said substance through a transdermal or trans-tissue window or port.

10. The system of claim 1 wherein the monitored person is not identified by the inventive system itself because one of: a) someone chooses not to despite having the choice or capability, b) software chooses not to, or c) identification means are not included as part of the inventive system.

11. The system of claim 1 wherein the monitored person is identified by other means, including: a) the patient's own input, b) a practitioner's, doctor's or clinician's input, c) a technician's input, d) input from a patient tracking or identification means not an integrated physical part of the inventive system.

12. The system of claim 1 with or without system-included identification means for identifying the patient, wherein the labeling approach or media utilizes at least one of:
a) one or more nanoparticles with or without a controlled size, shape or compositional parameter;
b) one or more microparticles with or without a controlled size, shape or compositional parameter;
c) one or more quantum dots with or without a controlled size, shape or compositional parameter;
d) one or more fluorophores, phosphors or chromophores;
e) one or more absorbing, adsorbing, attenuating, diffracting or polarizing materials;
f) one or more electrically chargeable or magnetic materials;
g) one or more micromachined, lithographically-derived or micromolded-derived materials or particles;
h) one or more particles having a biologically functional coating or entity on, in or under its surface;
i) one or more biologically or chemically targeted particles or materials;
j) one or more carbon nanotubes or buckeyballs;
k) one or more providers of a spectral or signal background from which labels subtract-from, modulate, or mask said background signature or signal;
l) an additive label;
m) a subtractive label;
n) a label which is read using two or more energy fluxes or fields;
o) a label which is read using acoustics or acoustic cavitation;
p) a label which is read in the visible, infrared or x-ray wavelengths;
q) a label which is read by a polarization detection or quantification means;
r) a label which is read using any excitation or biasing field or energy;
s) a label not requiring excitation or field-exposure for reading;
t) a label providing information on a drug concentration presently chemically or metabolically available to the body;
u) a label providing information on a drug concentration which can potentially become chemically or metabolically available to the body;
v) a label providing information on a drug concentration which has been utilized by the body or already made available to the body-whether chemically or metabolically altered or not;
w) a label utilizing one or more selected isotopes of one or more elements;
x) a label utilizing a controlled ratio of isotopes, fluorophores or chromophores;
y) any label having a unique decay parameter such as an optical decay time; or
z) any label that exhibits a nuclear emission or decay signature.

13. The system of claim 1 wherein the system provides for any one or more of:
a) home use;
b) ambulatory use;
c) enforced use;
d) location-tracking of the patient or a usage event or noncompliance;
e) bed-use;
f) self-use;
g) automated operation;
h) physically remote monitoring across a physical distance but in the same general location-as monitoring optically or using Bluetooth® wireless connection from across the room using light waves or radio waves passing across said room;
i) monitoring over a network, wireless or wired;
j) recording of compliance or noncompliance;
k) trend recording or statistics generation;
l) corrective-action feedback to the patient whether by software or by a human practitioner or both;
m) authorization of drug dispensing or prescriptions;
n) discrete use such a system as when it appears to only be a cell phone or PDA;
o) control of a drug depot such as a pump, patch or implanted storage entity; or
p) adjustment of recommended dose or ingestion based on the physical or mental state of the patient or subject.

14. The system of claim 1 wherein the system signature-detection means is split into two or more portions, with at least one portion mounted on or in the patient tissues and at least one other portion which can be otherwise carried or located nearby—not necessarily carried or on the body.

15. The system of claim 14 wherein at least two sensing portions are connected, at least occasionally, by one or more of:
a) a cable or wired connection;
b) a wireless electrical or optical connection;
c) a placement of one portion in proximity or contact with the other at least momentarily;
d) a temporary physical contacting of the two said portions; or
e) the plugging-in of a connectorized pluggable entity.

16. The system of claim 1 wherein patient-identification includes one or more of: a) identification by true name, b) identification by an alphanumeric or numeric identifier, c) identification by a nontrue alias, “handle” or substitute name, d) identification as being one of a specific trait—such as male, female, trustworthy, nontrustworthy, e) identification as being a person for which prior data has been taken using an inventive system or the particular inventive system, f) identification as being in or from a specific location or area, g) identification as by storage and recognition of any biometric signature—such as a vein pattern, h) identification as a substance abuser, i) identification as someone in potential danger of a bad drug interaction, j) identification which triggers or is enabled or authorized by the reading-of or writing-to a medical record, or k) identification that involves relating or reporting a prescription or diagnosis to that particular patient or to his/her caregiver.

17. The system of claim 1 wherein the system is one of:
a) voluntarily worn or used by the patient or subject;
b) involuntarily worn or used by the patient or subject;
c) locked upon the patient's body; or
d) worn under the clothing.

18. The system of claim 1 further including at least one excitation filter and at least one emission filter to respectively stimulate and detect fluorescence from ingesting fluorophores and/or chromophores.

19. A method for monitoring drug therapy compliance by a human or animal patient consisting of a device that can detect a signal from a labeled pill, chewable capsule, lozenge, suppository, ingestible tablets, buccal tablets, troches, elixirs, suspensions, syrups, wafers, and the like administered to the patient and determining from the presence or absence of said detection whether said patient has ingested or received said pill, capsule or liquid in compliance with the medication regimen.

20. The method of claim 19 wherein said pill, capsule, lozenge, suppository or liquid is a therapeutic substance selected from the group consisting of agents for the common cold, anti-addiction, anti-infectives, analgesics, anesthetics, anorexics, antiarthritics, anti-allergy agents, antiasthmatic agents, anticonvulsants, anti-depressants, antidiabetic agents, anti-depressants, anti-diuretics, anti-emetics, anti-histamines, anti-inflammatory agents, antimigraine preparations, antimotion sickness preparations, antinauseants, antineoplastics, anti-obesity, antiosteoporeteic, anti-parkinsonism drugs, antipruritics, antipsychotics, antipyretics, anticholinergics, benzodiazepine antagonists, bone stimulating agents, central nervous system stimulants, hormones, hypnotics, immunosuppressives, prostaglandins, proteins, peptides, polypeptides and other macromolecules, psychostimulants, rhinitis treatment, sedatives, sexual hypofunction, vaccines, tranquilizers, nutrients vitamins, minerals, herbs, and water.

21. The method of claim 19 wherein the device consists of an NIR source which either backlights the general target area or illuminates it reflectively or transmissively, thereby providing an optical contrast or an optical indication of a portion of the vasculature.

22. The method of claim 19 wherein a camera or detector is utilized and that camera or detector includes at least one of a) a CMOS chip, b) a CCD chip, c) a chip having both visible and infrared sensitivity, d) a photodetector chip designed for a specific wavelength range such as mid-IR, near-IR or infrared in general.

23. The method of claim 19 wherein the observer
(a) looks at a digital or analog readout or a set of lights related to the presence or absence of a specific fluorophore signal;
(b) looks through an optical window, filter, lens or shutter;
(c) listens to an audible feedback signal which is related to the presence or absence of a specific fluorophore signal; or
(d) feels a tactile feedback signal which is related to the presence or absence of a specific fluorophore signal.

24. The method of claim 19 wherein a reader is present which serves to interpret the type and strength of signal received in order to determine specific parameters such as the name of the drug which was taken, its concentration, and the sex and/or name of the person intended to receive the medication.

25. The method of claim 24 wherein a kit is provided which contains a reservoir of a specific fluorophore in the form of a dropper or spray, which the patient can use to apply the fluorophore to their pills for the purposes of identification in order to differentiate them from another person's pills.

26. The method of claim 25 wherein multiple fluorophores or chromophores are detectable with a light that causes fluorescence, one of said fluorophores or fluorophore combinations causing a different fluorescent coloration of a portion of the vasculature than the fluorescent coloration caused by other fluorophores or fluorophore combinations.

27. The method of claim 24 wherein a kit is provided that contains a reservoir of a specific fluorophore in the form of a dropper or spray, which a third person can administer to the food of an anorexic or bulimic patient in order to determine whether the food was actually ingested and absorbed into the blood stream.

28. The method of claim 24 wherein a kit is provided that contains a master chart or template so that the observer can interpret the signal received by matching its color or intensity to a specific parameter on the chart.

29. The method of claim 24 wherein a timer unit is present which is continuously running and internally maintains the current time, the timer optionally being provided with a memory to store the viewing times for later retrieval.

30. The method of claim 19 wherein said composition is a medication or medication composition.

31. The method of claim 19 which consists of visually observing the vasculature of said patient to determine the presence or absence of a fluorophore signal.

32. The method of claim 19 wherein the pill is labeled with a fluorophore, specific concentration of a fluorophore(s) or combination of fluorophores in order to provide a drug-specific signature or drug dose estimation.

33. The method of claim 32 wherein said fluorophore or fluorophores include any one or more of rhodamine, indocyanine-green (IcG), fluoroscein, phenyl alanine, tyrosine, tryptophan, retinol, riboflavin, pryidioxin, A-tocopherol, NADH, ATP, chlorophyll-a, hematoporphyrin, or quinine.

34. The method of claim 32 wherein one of said fluorophores causes a different coloration or intensity than another of said fluorophores.

35. The method of claim 19 further comprising providing multiple fluorophores in combination wherein one of said fluorophore combinations results in a different coloration or coloration pattern in the vasculature than another of said fluorophore combinations.

36. The method of claim 19 further comprising providing multiple fluorophores in combination, causing coloration detectable with a light which causes fluorescence which is visually observable for determining whether said patient has ingested said combination in compliance with a medication regimen and for determining a time frame in which the combination was ingested.

37. The method of claim 19 wherein the pill is labeled with a ferromagnetic or paramagnetic substance detectable with a coil as in NMR and other detection methods in order to provide a drug-specific signature or drug dose estimation.

38. The method of claim 19 wherein patient-identification includes one or more of: a) identification by true name, b) identification by an alphanumeric or numeric identifier, c) identification by a non-true alias, “handle” or substitute name, d) identification as being one of a specific trait—such as male, female, trustworthy, nontrustworthy, e) identification as being a person for which prior data has been taken using an inventive system or the particular inventive system, f) identification as being in or from a specific location or area, g) identification as by storage and recognition of any biometric signature—such as a vein pattern, h) identification as a substance abuser, i) identification as someone in potential danger of a bad drug interaction, j) identification which triggers or is enabled or authorized by the reading-of or writing-to a medical record, or k) identification which involves relating or reporting a prescription or diagnosis to that particular patient or to his/her caregiver.

39. The method of claim 19 wherein the system is one of:
a) voluntarily worn or used by the patient or subject;
b) involuntarily worn or used by the patient or subject;
c) locked upon the patient's body; or
d) worn under the clothing.

40. A database or storage means for at least one patient including:
a) patient identity or tracking information, including a name or biometric signature; or
b) patient monitoring data or useable memory space for such for at least one patient ingested or ingestable substance,
the patient wearing, carrying, presenting oneself to or having a monitoring system capable of both said monitoring and said identification, the database being anywhere including being remotely located and/or on the patients system.

41. The database or storage means of claim 40 wherein at least one of:
a) patient data is transferred over a wired or wireless network;
b) patient data is compared to the data of a population of patients or subjects;
c) patient date is statistically analyzed at any point or any location;
d) patient data is reported to anyone in any manner;
e) patient data is contained in a controlled-access database;
f) patient data includes data relating to potential drug interactions;
g) patient data includes data relating to the health state of the patient; or
h) patient data is sampled over time, whether automatically or manually.

42. The database or storage means of claim 40 wherein the monitoring system is one of:
a) voluntarily worn or used by the patient or subject;
b) involuntarily worn or used by the patient or subject;
c) locked upon the patient's body; or
d) worn under the clothing.

43. Apparatus for detecting a fluorescent signature from at least one ingested fluorophore, the apparatus comprising:
a collimated light source that emits electromagnetic radiation in the UV to blue region;
an excitation filter through which the electromagnetic radiation is passed;
a dichroic mirror that passes relatively shorter wavelengths and reflects relatively longer fluorescent wavelengths;
means for directing the first electromagnetic radiation onto an area of a body containing the at least one ingested fluorophore to thereby cause fluorescent radiation to be emitted from the at least one ingested fluorophore;
means for receiving the fluorescent radiation, the fluorescent radiation being longer than the electromagnetic radiation and thereby reflected off the dichroic mirror;
an emission filter through which the fluorescent radiation is passed; and
an optical detector for detecting the fluorescent radiation.

44. The apparatus of claim 43 further including a power supply for providing power to the collimated light source and the optical detector.

45. The apparatus of claim 43 wherein the excitation filter is centered on 417 nm, with a bandwidth of 60 nm.

46. The apparatus of claim 43 wherein the emission filter is centered on 697 nm, with a bandwidth of 75 nm.

47. The apparatus of claim 43 wherein the at least one fluorophore is selected from the group consisting of rhodamine, indocyanine-green (IcG), fluoroscein, phenyl alanine, tyrosine, tryptophan, retinol, riboflavin, pryidioxin, A-tocopherol, NADH, ATP, chlorophyll-a, hematoporphyrin, and quinine.
